(19) United States
(12) Patent Application Publication
(10) Pub. No.: US 2004/0254427 A1
(43) Pub. Date: Dec. 16, 2004

(54) Surgical stay-grip assembly and method of using same

(75) Inventor: James M. Fowler JR., Houston, TX (US)

Correspondence Address: ANDREWS & KURTH, L.L.P., 600 TRAVIS, SUITE 4200, HOUSTON, TX, 77002, US

(73) Assignee: Lone Star Medical Products, Inc., Houston, TX (US)

(21) Appl. No.: 10/462,170

(22) Filed: Jun. 16, 2003

(51) Int. Cl. A61B 1/32

(52) U.S. Cl. 600/210

(57) ABSTRACT

A surgical stay-grip assembly includes a pair of jaws pivotally connected to one another and a jaw closing member, preferably a spring, for urging a first end of the jaws together. An elongated member, preferably elastic, is connected to one of the jaws. One jaw includes a receiver for securing the elongated member to the jaw. The other jaw preferably includes a receptacle adapted to secure an elongated member to this other jaw. When securing to the receptacle, the elongated member includes an enlarged end portion and the receptacle includes an opening adapted to retain the enlarged end portion within the receptacle. Preferably, the surgical stay-grip assembly includes a retractor hook assembly connected to the distal end of the elongated member.

### CROSS-REFERENCE TO RELATED APPLICATIONS

Not applicable.

### STATEMENTS REGARDING FEDERALLY SPONSORED RESEARCH OR DEVELOPMENT

Not applicable.

### REFERENCE TO A MICROFICHE APPENDIX

Not applicable.

### BACKGROUND OF THE INVENTION

1. Field of the Invention

The present invention relates to surgical retractor stays, and more particularly to an improved grip assembly for a surgical retractor stay that can be used in surgery for retracting tissue at an incision.

2. Description of the Related Art

Many types of surgeries have surgical sites that require several retractor stays to hold the incision open. In some situations, the retractor must necessarily include a very small stay for holding the incision open in a restricted area. Such a retractor stay construction is shown in U.S. Pat. No. 4,430,991, issued to Darnell and entitled “Surgical Retractor Stay Device And Tube Connector.” The Darnell patent provides a frame that accepts one end portion of an elastic tubular member. The other end of the tubular member carries a hook construction for engaging the selected tissue.

In some surgical procedures, the particular geometry presented does not allow for the use of a retractor ring of the type shown in the Darnell patent at the wound site. However, the elastic stay that is shown in the Darnell patent continues to provide benefit to the surgeon even when the retractor ring cannot be fitted to the wound site. Currently, surgeons are using the elastic stays shown in the Darnell '991 patent by clamping the tail end of the elastic stay to a surgical drape using a hemostat or forceps. In order to clamp the elastic stay tail of the Darnell patent type of stay using a hemostat or forceps, such could require up to three hands. One hand is needed to hold the hemostat or forceps. One hand is needed to pull the elastic stay tail. Yet a third hand must position the drape.

Another type of device that is commonly used in surgical operations to grasp tissue and lift or pull the tissue out of the way is an Allis clamp, available from various manufacturers. The Allis clamp is unlocked in its natural state. When attached to tissue, the Allis clamp is locked by squeezing hemostat or forceps-like handles together to engage a tooth and cog mechanism near the handles. The tighter the handles are squeezed, the further in the tooth and cog mechanism ratchets and the tighter the clamp end grips the tissue. A disadvantage of the Allis clamp arises when several of the devices are used around a wound site. In such instances, the several hemostat or forceps-like handles placed around the wound site substantially clutter the operating area or surgical site.

Applicant's U.S. Pat. No. 5,899,853 discloses a surgical retractor having a pair of jaws urged together in a “closed” position by a jaw closing member provided by a receptacle at one end of an elongated elastic member. A portion of the elastic member is positioned in between the two jaws to provide elasticity that urges the jaws to clamp the jaw together. A retractor hook portion is connected to the distal end portion of the elongated elastic member.

It is desirable to have a gripping device for a surgical retractor stay that is compact, adaptable for use with tubing or other retractor stays, provides secure gripping of tissue, and closes automatically.

### BRIEF SUMMARY OF THE INVENTION

The preferred embodiment of the stay-grip assembly of the present invention includes a clamping assembly having a pair of jaws adapted to grip a material, such as a surgical drape, and a surgical stay connected to the clamping assembly for retraction, such as retracting tissue from an incision or open wound. The clamping assembly, in its natural state, is locked or closed under a fixed spring tension. The stay-grip assembly is very versatile and adapted for use with a variety of surgical stays or elongated members. The amount of jaw closure force in the clamping assembly can be varied by utilizing various spring installations. Releasing or opening the clamping assembly requires pressure applied to the jaw handles. Releasing the pressure to the jaw handles allows the jaw teeth to close or “bite.”

The preferred embodiment of the stay-grip assembly is adapted for use with a variety of stays, typically comprising elongated members, including, but not limited to, solid and hollow tubular members. The clamping assembly of the preferred embodiment of the present invention includes a stay receptacle and a stay receiver for attachment of various surgical stays and/or tubing for retraction. A stay-grip assembly can be attached to one or both ends of the elongated member. Preferably, the elongated member is an elastic member. Additionally, the stay-grip assembly is adapted for use with a surgical stay having some form of hook assembly attached to the distal end of the stay.

The preferred embodiment of the present invention includes a finger grip assembly having first and second finger grips adapted to slide onto handle portions of the jaws. The finger grip assembly prevents slippage under wet or slippery conditions. Preferably, the finger grip assembly includes a skirt extending between the first and second finger grips. The skirt is formed by a pair of walls extending between the first and second finger grips at the rear corners of the clamping assembly. An open space is defined between the skirt walls and the rear portion of the first and second finger grips. The open space is provided to center and maintain the stay extending from the rear of the clamping assembly. The skirt also prevents suture cords and other surgical apparatus from being caught between the handle portions. The skirt walls are pliable and generally collapse upon the handle portions being squeezed together.

### BRIEF DESCRIPTION OF THE SEVERAL VIEWS OF THE DRAWINGS

For a further understanding of the nature, objects, and advantages of the present invention, reference should be had to the following detailed description, read in conjunction with the following drawings, wherein like reference numerals denote like elements and wherein:

FIG. 1 is a top view of a surgical stay-grip assembly according to a preferred embodiment of the present invention, the stay-grip assembly shown retracting tissue at an incision,

FIG. 2 is a perspective view of an embodiment of the present invention;

FIG. 3 is a view taken along lines 3-3 of FIG. 2;

FIG. 4 is a view taken along lines 4-4 of FIG. 3;

FIG. 5 is a view taken along lines 5-5 of FIG. 3;

FIG. 6 is partial sectional, elevation view of the stay-grip assembly of FIG. 2, showing alternative elastic stays and connections to the clamping assembly;

FIG. 7 is a lower, front right perspective view of the first jaw; and

FIG. 8 is an upper, front right perspective view of the second jaw.

### DETAILED DESCRIPTION OF THE INVENTION

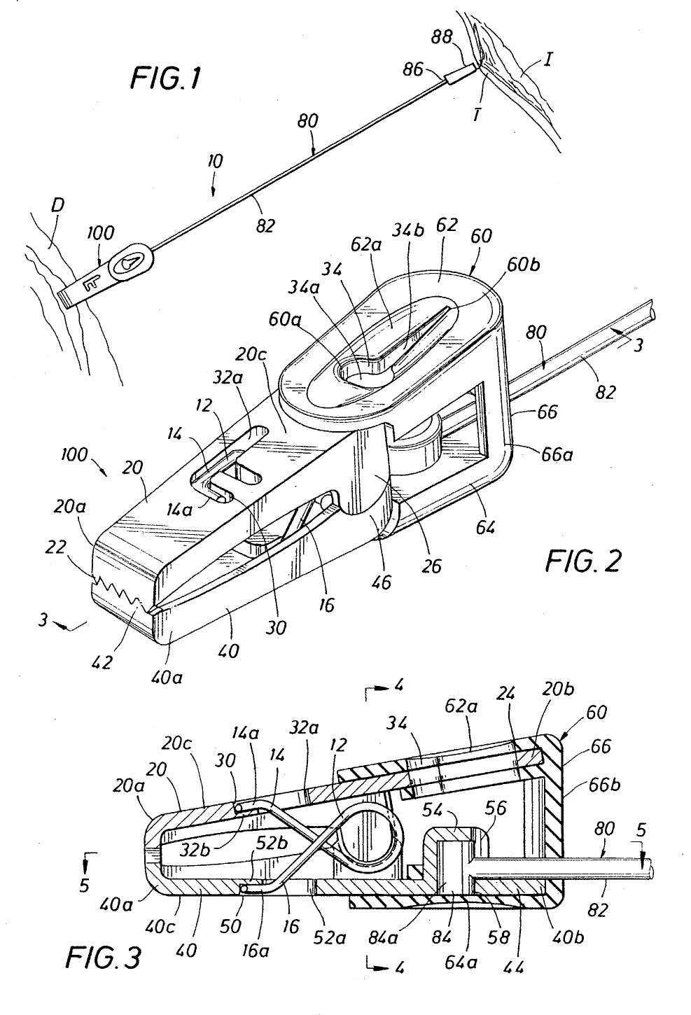

A preferred embodiment of the stay-grip assembly of the present invention, designated generally as 10, is shown in FIG. 1. The stay-grip assembly 10 is shown retracting tissue T at an incision I. The preferred embodiment of the stay-grip assembly 10 includes a clamping assembly 100 having first and second jaws 20 and 40 (FIG. 2), respectively, and a stay 80 (FIG. 1). In the preferred embodiment of the present invention, a jaw closing member or spring 12 facilitates a hinged connection of the jaws 20 and 40 while also providing a jaw closure force as will be explained below. As shown in the embodiment illustrated in FIG. 1, the stay-grip assembly 10 retracts the tissue T by attaching the clamping assembly 100 to a surgical drape D or other member with the stay 80 connecting the clamping assembly 100 to a hook assembly 88.

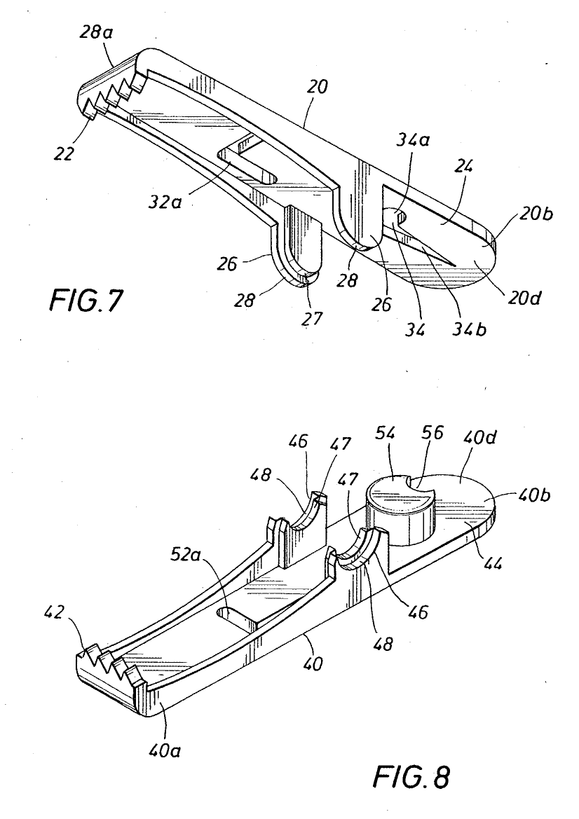

Referring to FIGS. 2 and 7, the first jaw 20 includes a serrated gripping surface or teeth 22, preferably at or near one end 20a of the first jaw 20. The second jaw 40 preferably includes a complementary serrated gripping surface 42 at or near one end 40a of the second jaw 40 as shown in FIGS. 2 and 8. A distal end 20b of the first jaw 20 includes a handle portion 24 (FIG. 7) and a distal end 40b of the second jaw 40 includes a handle portion 44 (FIG. 8). The handle portions 24 and 44 are adapted to be gripped by a user's hand in order to open the complementary serrated gripping surfaces 22 and 42 of the jaws 20 and 40, respectively. The jaws 20 and 40 are preferably injection molded in a rigid material such as polycarbonate.

As shown in FIGS. 2 and 6, the two jaws 20 and 40 are connected together, preferably via a pivot or hinge connection. In the preferred embodiment, the first jaw 20 includes a pair of hinge segments 26 with each hinge segment 26 having an articulating surface 28, preferably a convex articulating surface, as shown in FIG. 7. The second jaw 40 includes a pair of receptacle segments 46 with each receptacle segment 46 having a complementary articulating surface 48, preferably a concave articulating surface, as shown in FIG. 8. The engagement of the hinge segments 26 with the receptacle segments 46 allows pivotal movement of the jaws 20 and 40.

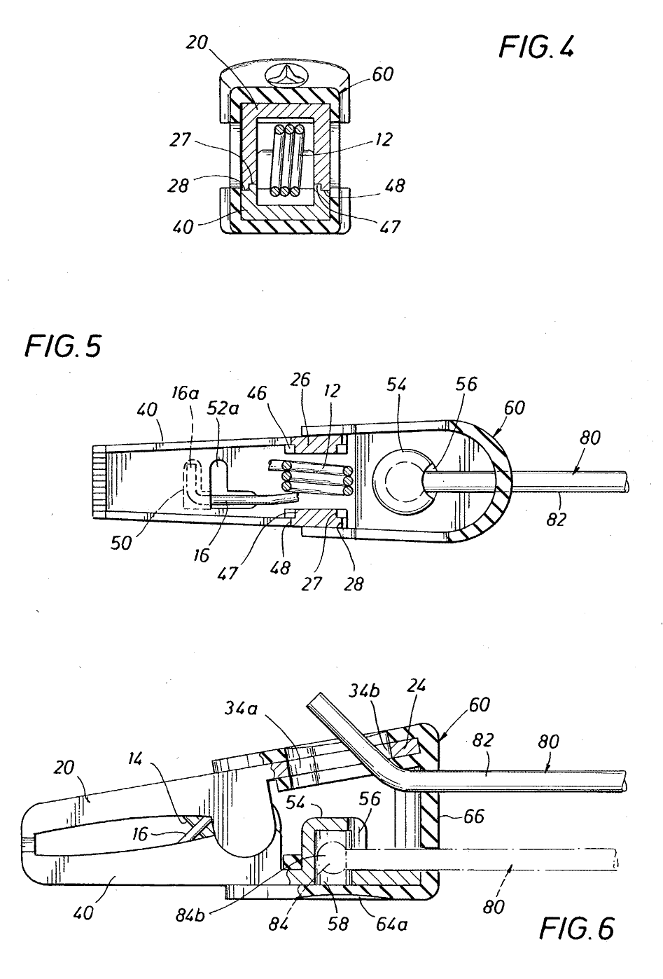

Referring to FIGS. 7 and 8, in the preferred embodiment of the present invention, the hinge segments 26 and the receptacle segments 46 include complementary guide surfaces 27 and 47, respectively. As shown in FIG. 7, the hinge segment 26 and guide surface 27 form a “step” and the receptacle segment 46 and the guide surface 47 form a complementary step as shown in FIG. 8. As shown in FIGS. 4 and 5, the cooperating hinge and receptacle segments 26 and 46 along with the cooperating guide surfaces 27 and 47 form a cooperating “step” which maintains relative alignment of the jaws 20 and 40 in addition to providing pivotal movement therebetween. Maintaining the relative alignment of the jaws 20 and 40 assures proper meshing of the teeth 22 and 42 when the jaws 20 and 40 are closed.

Referring to FIGS. 2 and 3, the spring 12 includes first and second arms 14 and 16, respectively. The spring 12 is preferably a torsion spring and preferably made of stainless steel. As shown in FIGS. 3 and 5, the coils of the torsion spring 12 are positioned between the cooperating pairs of hinge and receptacle segments 26 and 46. The jaws 20 and 40 include arm attachment portions 30 and 50, respectively, as shown in FIG. 3. In the preferred embodiment of the present invention, the arm attachment portions 30 and 50 include a slotted portion 32a and 52a, respectively extending through the jaws 20 and 40 as shown in FIG. 3. The arm attachment portions 30 and 50 further include, in the preferred embodiment, a stop portion 32b and 52b, respectively, which first and second arm end portions 14a and 16a, respectively, are maintained in contact with due to the spring force. Preferably, the stop portions 32b and 52b are recessed as shown in FIG. 3 so that the spring arm end portions 14a and 16a are maintained flush or slightly below the outer surface 20c and 40c, respectively, of the jaws 20 and 40.

Referring to FIGS. 3 and 8, a stay receptacle 54 is attached to an inner surface 40d of the handle portion 44. The stay receptacle 54 includes a first opening 56 through which a portion of a stay 80, preferably an elastic stay, is capable of extending. The stay receptacle 54 preferably includes a second opening 58 (FIGS. 3 and 6) extending through the handle portion 44 of the second jaw 40.

In the preferred embodiment, the stay 80 has an elongated portion 82, preferably elastic. In one embodiment of the present invention, the stay 80 has an enlarged end portion 84. As shown in FIG. 3, the stay 80 includes a cylindrical end portion 84a mounted transverse to the elongated portion 82. The cylindrical end portion 84a is larger than the opening 56 to prevent the end portion 84a from disengaging from the clamping assembly 100. Preferably, the end portion 84a can be inserted into the stay receptacle 54 through the first opening 56. However, it is to be understood that some stays 80 may be inserted into the stay receptacle 54 by inserting the elongated portion 82 through the second opening 58 and then the first opening 56 and pulling it through until the enlarged end portion 84 is received in the stay receptacle 54. The enlarged end portion 84 may include a generally spherical end portion 84b as shown in phantom in FIG. 6. It is to be understood that the cylindrical and spherical shaped end portions 84a and 84b, respectively, are preferred shapes, however various shapes of the enlarged end portion 84 are within the scope of the present invention.

Referring to FIGS. 2, 6 and 7, the handle portion 24 of the first jaw 20 preferably includes a stay receiver 34 adapted to secure a stay 80 to the clamping assembly 100. The stay receiver 34 as shown in FIGS. 2 and 7 includes an enlarged forward opening 34a joined to a rearwardly tapered slot 34b. The stay 80 can be secured to the clamping assembly 100 by passing the end of the stay elongated portion 82 through the enlarged forward opening 34a. If the stay 80 is an elastic stay having a generally uniform cross section, the elongated portion 82 can be secured in the tapered slot 34b by pulling rearwardly on the stay 80, causing deformation of the elastic stay 80 in the tapered slot 34b. In this embodiment of the stay-grip assembly 10, the length of the stay 80 between a distal end 86 (FIG. 1) and the clamping assembly 100 is infinitely adjustable along the length of the stay 80. Alternatively, if the stay 80 has an enlarged end portion 84, as for example a spherical shaped end portion 84b as shown in phantom in FIG. 6, the tapered slot 34b can be sized to receive the elongated portion 82 while preventing the enlarged end portion 84 from passing.

Referring to FIGS. 2 and 3, the preferred embodiment of the present invention includes a finger grip assembly 60 having first and second finger grips 62 and 64 adapted to slide onto the handle portions 24 and 44 of the jaws 20 and 40, respectively. Preferably, the finger grip assembly 60 is made of silicone and is preferably molded. The finger grip assembly 60 prevents slippage under wet or slippery conditions. As shown in FIG. 2, the first and second finger grips 62 and 64 preferably include a concave finger grip area 62a and 64a to further aid in the prevention of finger slippage under wet or slippery conditions. It is to be understood that the concave finger grip area may alternatively be placed on one of the two finger grips 62 and 64. It is to be further understood that concave finger grip areas could alternatively be included on the handle portions 24 and 44 in embodiments in which a finger grip assembly 60 is not used.

Additionally, as best seen in FIG. 2, the finger grip assembly 60 preferably includes openings 60a and 60b corresponding to the openings 34a and 34b, respectively, in the handle portion 24 of the jaw 20. This allows the stay 80 to extend through the openings 34b and 60b if connected to the stay receiver 34.

Preferably, the finger grip assembly 60 includes a skirt 66 extending between the first and second finger grips 62 and 64 as shown in FIGS. 2, 3 and 6. The skirt 66 is formed by a pair of walls 66a (FIG. 2) and 66b (FIG. 3) extending between the first and second finger grips 62 and 64 at the rear corners of the clamping assembly 100. An open space is defined between the walls 66a and 66b of the skirt 66 and the rear portion of the first and second finger grips 62 and 64. With reference to FIGS. 2 and 3, the open space between the skirt walls 66a and 66b is provided to center and maintain the stay 80 extending from the rear of the clamping assembly 100. The skirt 66 also prevents suture cords and other surgical apparatus from being caught between the handle portions 24 and 44. It is to be understood that the skirt walls 66a and 66b are pliable and generally collapse upon the handle portions 24 and 44 being squeezed together.

As discussed above, the surgical stay-grip assembly 10 includes a stay 80 connected to the clamping assembly 100. It is to be understood that elongated portion 82 of the stay 80 is preferably elastic and may be tubular or solid. As shown in FIG. 1, the stay 80 includes a distal end 86. The distal end 86 is shown connected to a hook assembly 88. The hook assembly 88 may comprise any of a variety of shapes and configurations as would be apparent to one of ordinary skill in the art. Alternatively, the distal end 86 may be adapted to connect with another clamping assembly 100 in which case the distal end 86 may have an enlarged end portion 84 or a generally uniform cross section as shown in FIG. 6.

The surgical stay-grip assembly 10 of the present invention includes many desirable features as discussed above assemblies 88 known to those of skill in the art that could be used with the present invention. The preferred embodiment of the present invention, in its natural state, is locked or closed under a fixed spring tension. The amount of jaw closure force can be varied by utilizing various spring installations in the stay-grip assembly 10. Preferably, the spring force is in the range of one to five pounds. Preferably, a stainless steel torsion spring 12 is used to provide the spring gripping force. Releasing or opening the stay-grip assembly 10 requires pressure applied to the jaw handles 24 and 44. Releasing the pressure to the jaw handles 24 and 44 allows the teeth or jaw serrated gripping surfaces 22 and 42 to close or “bite.” The jaws 20 and 40 can grip the drape or other material D that surrounds the incision or wound opening I when the jaws 20 and 40 are manipulated by a user's hand. Due to the nature of its use, the stay-grip assembly 10 is preferably used only once and then disposed of.

The preferred embodiment of the stay-grip assembly 10 is adapted for use with a variety of stays 80, typically comprising elongated members 82, including, but not limited to, solid and hollow tubular members. The preferred embodiment of the present invention includes a clamping assembly 100 having a stay receptacle 54 and a stay receiver 34 for attachment of various surgical stays 80 and/or tubing for retraction. A stay-grip assembly 10 can be attached to one or both ends of the elongated member 82. Preferably, the elongated member 82 is an elastic member. One suitable material for the elastic elongated member 82 is silicone. Additionally, the stay-grip assembly 10 is adapted for use with a surgical stay 80 having some form of hook assembly 88 attached to the distal end 86 of the stay 80. It is understood that there are a variety of surgical stay hook.

The description given herein is intended to illustrate a preferred embodiment of the present invention. It is to be understood that the present invention should not be unduly limited to the foregoing embodiment which has been set forth for illustrative purposes. Various modifications and alterations of the invention will be apparent to those skilled in the art, and such modifications and alterations may be made without departing from the true scope of the invention. Therefore, it is intended that all such variations be included within the scope of the present invention as claimed herein.

I claim:

1. A surgical stay-grip assembly comprising:
a first jaw having first and second ends;
a second jaw having first and second ends, said second jaw pivotally connected to said first jaw;
a jaw closing member for urging said first end of said jaws together; and
an elongated member having first and second ends, said first end of said elongated member connected to one of said jaws.

2. The surgical stay-grip assembly of claim 1, wherein said jaws are pivotally connected at cooperating concave and convex surfaces.

3. The surgical stay-grip assembly of claim 2, wherein said first jaw includes a convex articulating surface and said second jaw includes a concave articulating surface.

4. The surgical stay-grip assembly of claim 3, wherein said convex and concave articulating surfaces maintain alignment of said first jaw with said second jaw.

5. The surgical stay-grip assembly of claim 1, wherein said first end of said jaws abut in a closed position and are spaced apart in an open position.

6. The surgical stay-grip assembly of claim 1, wherein said jaw closing member is a spring.

7. The surgical stay-grip assembly of claim 6, wherein said spring is a torsion spring.

8. The surgical stay-grip assembly of claim 6, wherein said spring includes first and second arms, said first arm engaging said first jaw and said second arm engaging said second jaw.

9. The surgical stay-grip assembly of claim 6, wherein said spring maintains said jaws in pivotal relationship with each other.

10. The surgical stay-grip assembly of claim 1, wherein said first end of each said jaw includes a gripping surface.

11. The surgical stay-grip assembly of claim 10, wherein each said gripping surface includes serrated teeth.

12. The surgical stay-grip assembly of claim 1, wherein said first jaw includes a receiver for securing said elongated member to said first jaw.

13. The surgical stay-grip assembly of claim 12, wherein said receiver is adapted to releasably secure said elongated member to said first jaw.

14. The surgical stay-grip assembly of claim 12, wherein said receiver comprises an opening in said first jaw, said opening adapted to secure said elongated member to said first jaw.

15. The surgical stay-grip assembly of claim 14, wherein said receiver opening includes a tapered slot adapted to engagingly secure said elongated member to said first jaw.

16. The surgical stay-grip assembly of claim 1, wherein said second jaw includes a receptacle for securing said elongated member to said second jaw.

17. The surgical stay-grip assembly of claim 16, wherein said first end of said elongated member includes an enlarged end portion and said receptacle includes an opening adapted to retain said enlarged end portion within said receptacle.

18. The surgical stay-grip assembly of claim 1, further comprising a skirt extending between said second end of said jaws, said skirt including a skirt opening through which said elongated member extends, said skirt opening adapted to positionally maintain said elongated member.

19. The surgical stay-grip assembly of claim 1, further comprising a finger grip assembly adapted to attach to said second end of said jaws, said finger grip assembly providing a non-slip surface.

20. The surgical stay-grip assembly of claim 19, wherein said finger grip assembly includes a concave finger grip area.

21. The surgical stay-grip assembly of claim 12, wherein said second jaw includes a receptacle for securing said elongated member to said second jaw.

22. The surgical stay-grip assembly of claim 1, further comprising a retractor hook assembly connected to said second end of said elongated member.

23. The surgical stay-grip assembly of claim 22, wherein said elongated member is elastic.

24. A surgical stay-grip assembly comprising:
a pair of jaws, each jaw having first and second ends, said pair of jaws pivotally connected to one another;
a jaw closing member for urging said first end of said jaws together; and
an elongated, elastic member having first and second ends, said first end of said elongated member releasably connected to one of said jaws.

25. The surgical stay-grip assembly of claim 24, wherein said jaws pivot one upon the other at cooperating concave and convex articulating surfaces.

26. The surgical stay-grip assembly of claim 25, wherein said convex and concave articulating surfaces maintain alignment of one said jaw with the other said jaw.

27. The surgical stay-grip assembly of claim 24, wherein said jaw closing member is a torsion spring.

28. The surgical stay-grip assembly of claim 24, wherein said first end of each said jaw includes a gripping surface.

29. The surgical stay-grip assembly of claim 24, wherein one said jaw includes a receiver for securing said elongated, elastic member to said jaw.

30. The surgical stay-grip assembly of claim 29, wherein said receiver comprises an opening in the one said jaw, said opening adapted to secure said elongated, elastic member to the one said jaw.

31. The surgical stay-grip assembly of claim 30, wherein said receiver opening includes a tapered slot adapted to engagingly secure said elongated, elastic member.

32. The surgical stay-grip assembly of claim 29, wherein the other of said jaws includes a receptacle adapted to secure said elongated, elastic member to said jaw.

33. The surgical stay-grip assembly of claim 32, wherein said first end of said elongated, elastic member includes an enlarged end portion and said receptacle includes an opening adapted to retain said enlarged end portion within said receptacle.

34. The surgical stay-grip assembly of claim 24, further comprising a retractor hook assembly connected to said second end of said elongated, elastic member.

35. A method of assembly of a surgical retractor for retracting tissue during a surgical procedure, the surgical retractor including a clamping assembly and one of a plurality of hook assemblies, each hook assembly having an elongate, elastic member connected thereto, the method comprising the steps:
selecting the desired hook assembly for the surgical procedure; and
releasably securing the elongate, elastic member to the clamping assembly;
wherein the clamping assembly includes a pair of gripping jaws which are spring-biased in a closed position independent of the elongate elastic member.

36. The method of claim 35, wherein the step of releasably securing comprises a first receiver in the clamping assembly for receiving a portion of the elongate, elastic member.

37. The method of claim 35, further comprising the step of selecting between alternative means for securing the elongate, elastic member to the clamping assembly.

38. The method of claim 35, further comprising the step of attaching a skirt to the clamping assembly to maintain the elongate, elastic tail in a rearward direction from the clamping assembly.

39. The method of claim 35, further comprising the step of selecting a clamping force of the clamping assembly by selecting a spring of suitable spring force.
